(19) United States
(12) Patent Application Publication
(10) Pub. No.: US 2011/0230788 A1
(43) Pub. Date: Sep. 22, 2011

(54) Methods And Systems For Sensing On Body Of Patient

(76) Inventors: Sandeep Venkit Anand, Bangalore (IN); Debiprosad Roy Mahapatra, Bangalore (IN)

(21) Appl. No.: 12/726,111

(22) Filed: Mar. 17, 2010

(51) Int. Cl. A61B 5/103

(52) U.S. Cl. 600/587

(57) ABSTRACT

Methods and systems for identifying a strain history of a portion of a body of a patient are disclosed. The method includes measuring an electrical response of at least one thin-film sensor of a sensor apparatus that is applied to the portion of the body of the patient to obtain a reference signal. The at least one thin-film sensor includes an electrically resistant material, conductive nanoparticles dispersed substantially throughout the electrically resistant material, and conductive nano-structures dispersed substantially throughout the electrically resistant material. The electrical response of the at least one thin-film sensor is monitored to detect changes in the electrical response. Based on the changes in the electrical response, a strain history of the at least one thin-film sensor is determined. A strain history for the portion of the body of the patient is identified based on the strain history of the at least one thin-film sensor.

### BACKGROUND

Strain may be defined as deformation experienced by a body resulting from application of a force. Sensors may be used for detecting strain behavior in the human back, human joints, and other critical points on a human body. The strain may be, for example, caused by various forces acting on the human body. These various forces acting on the human body may be forces such as tensile forces, compressive forces, and torsional forces. By identifying the strain behavior on a portion of the human body (e.g., a human joints), it may be possible to detect issues such as joint ailments (e.g., joint deterioration).

Existing ways of sensing strain include, for example, computational approaches, individual transducers, and pressure sensors, such as pressure-sensitive films and pressure-sensitive mats. Existing sensors include metallic-based or micro-electromechanical system (MEMs)-based strain-measuring devices. These sensors are generally of fixed sizes and fixed shapes. Also, these typical sensors are rigid and flat, and hence, may not be used for measuring strains on irregular and curved surfaces. Further, these existing sensors are relatively expensive and neither flexible nor machinable. For example, MEMS type semiconductor and fiber-optic strain sensors can achieve high sensitivities, but have high manufacturing costs and require costly data acquisition systems.

Commercially available constantan or nickel-chromium-alloy-based strain gages offer wide static, dynamic, and temperature ranges. However, these gages lack versatility and flexibility, as the gages may only measure strains at specific locations to which the gages are bonded and along a directional grid. In addition, the gages typically exhibit relatively low and narrow range of gauge factor, from 2.0 to 3.2.

The gauge factor of a strain sensor is defined as the relative change in the electrical resistance of the sensor for an applied mechanical strain. R₀ may be the resistance of the sensor under no strain condition, and the resistance may increase to R_{ε} under the application of a strain ε. Ignoring any temperature effects, the gauge factor, G, of that strain sensor may be given by the relationship:

$$G = \frac{( {R}_{ɛ} - {R}_{0} ) / {R}_{0}}{ɛ} = \frac{Δ R / {R}_{0}}{ɛ}$$

Gauge factor serves as an index of sensitivity of a sensor to mechanical strain. A higher gauge factor indicates more strain sensitivity. For example, the larger the gauge factor is, the smaller the strains that may be detectable by a sensor.

### SUMMARY

In one example aspect, a method for identifying a strain history of a portion of a body of a patient is described. The method includes measuring an electrical response of at least one thin-film sensor of a sensor apparatus that is applied to the portion of the body of the patient to obtain a reference signal. The at least one thin-film sensor includes an electrically resistant material, conductive nanoparticles dispersed substantially throughout the electrically resistant material, and conductive nano-structures dispersed substantially throughout the electrically resistant material. In addition, the at least one thin-film sensor has a gauge factor of greater than about 4. The method also includes monitoring the electrical response of the at least one thin-film sensor over a period of time to detect at least one change from the reference signal in the electrical response. Further, the method includes, based on the at least one change from the reference signal in the electrical response, determining a strain history of the at least one thin-film sensor. Still further, the method includes identifying a strain history for the portion of the body of the patient based at least on the determined strain history of the at least one thin-film sensor.

In another example aspect, a flexible sensor arrangement for identifying strain behavior for a portion of a body of a patient is described. The portion of the body of the patient may be, for example, a joint of the patient or a bone of the patient. The flexible sensor arrangement includes a plurality of thin-film sensors, wherein each of the thin-film sensors comprise an electrically resistant material, conductive nanoparticles dispersed substantially throughout the electrically resistant material, and conductive nano-structures dispersed substantially throughout the electrically resistant material. Further, the thin-film sensor has a resistivity that varies with a magnitude of strain applied to the thin-film sensor. In this flexible sensor arrangement, the plurality of thin-film sensors are arranged in a pattern for detecting strain behavior for the portion of the body of the patient. Example patterns include a grid-like pattern or a longitudinal array. It should be understood, however, that other patterns are possible as well.

In still another example aspect, a sensor apparatus is described that includes a plurality of thin-film sensors, a processing unit, and a wireless communication interface. The thin-film sensors comprise an electrically resistant material, conductive nanoparticles dispersed substantially throughout the electrically resistant material, and conductive nano-structures dispersed substantially throughout the electrically resistant material. Further, the thin-film sensor has a gauge factor of greater than about 4. Still further, the plurality of thin-film sensors are arranged in a pattern for detecting stress behavior for the portion of the body of the patient. The processing unit is configured to measure electrical resistance of each of the plurality of thin-film sensors. The wireless communication interface is in communication with the processing unit and is arranged to transmit data from the processing unit.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1(a) illustrates example thin-film sensors of an example sensor apparatus that may be attached to an example portion of a body of a patient;

FIG. 1(b) illustrates a block diagram of an example sensor apparatus and a block diagram of an example data acquisition and analysis system;

FIG. 2 illustrates a schematic diagram of an example sensor of FIG. 1(a) and example forces to which the sensor may be subjected;

FIG. 3 is a flowchart that depicts example steps of a method for identifying strain history of a portion of a body of a patient;

FIGS. 4(a)-(b) depict an example pattern suitable for detecting stress behavior for an example portion of the body of the patient;

FIGS. 4(c)-(d) depict an example pattern suitable for detecting stress behavior for an example portion of the body of the patient;

FIG. 5 depicts example thin film sensors consisting of a fabricated composite of multiwalled carbon nanotubes, epoxy, and carbon black, in film and wire forms, on glass and polycarbonate substrates;

FIG. 6 illustrates an example use of thin film sensor involving current measurement in a circuit upon static loading;

FIG. 7 is an example graph of the experimentally-determined DC current voltage characteristics of carbon black and epoxy composites, having 33% by volume carbon black, under no mechanical load;

FIG. 8 is an example graph of experimentally-determined DC current voltage characteristics of epoxy, carbon black, and carbon nanotube composites, having 33% by volume carbon black, and having various weight fractions of carbon nanotubes under no mechanical load;

FIG. 9 is an example graph of a comparison between measured and simulated resistance change as a function of strain for a sensor having 33% by volume carbon black and no bias voltage;

FIG. 10 is an example graph of simulated stress-strain curves for sensors having various volume fractions of carbon black at various bias voltages;

FIG. 11 is an example graph of experimentally-determined strain dependent resistance variations for sensors without an applied bias voltage and with different weight fractions of carbon nanotubes; and

FIG. 12 is a block diagram illustrating an example computing device.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and are made part of this disclosure.

A nano-structure material has structure on a molecular level. Fullerenes are examples of nano-structure. A fullerene is a molecule composed substantially (or in some examples entirely) of carbon atoms arranged in a particular shape, such as a hollow sphere, an ellipsoid, or a tube, for example. Carbon nanotubes (CNTs) are one example of cylindrical fullerenes.

Multiple features of CNTs make CNTs suitable for use in strain sensors. For example, CNTs may possess large surface areas, and an electrical conductivity of CNTs is a function of the chirality or composition of the nanotubes. A CNT also has a high Young's modulus under tensile force acting along a length of the CNT. (Young's modulus, E, is the stiffness of an isotropic elastic material.) CNTs may be subjected to forces without deforming and have sensitivity to changes in a surrounding environment. The tensile strengths of a wide variety of polymers may be enhanced by integrating of CNTs into the polymers, for example.

Another feature of CNTs may be an electronic energy band gap that increases with uniaxial and torsional strain. CNTs may typically undergo transition from a metallic state to a semiconducting state upon application of strain. Because electrons move more freely in a metallic state with zero energy band gap than in a semiconducting state with a higher electronic energy band gap, a metallic state may correspond to a lower resistivity than is present in a semiconducting state. Consequently, a resistance of CNTs typically increases when the CNTs are subjected to strain.

There are many types of CNTs. For example, one type includes single-walled carbon nanotubes (SWCNTs) that include hollow cylinders having walls that are a single-atom (of carbon) thick. Another type includes multiwalled carbon nanotubes (MWCNTs) that include either nested cylinders having walls that are a single-atom (of carbon) thick or rolls of a single-atom thick sheet (that would appear to be a spiral if observed end-on). Highly pure grade carbon nanotubes are those having greater than about 99% carbon by weight and less than about 1% impurities, such as metals.

Example embodiments describe methods and systems for identifying a strain history for a portion of a body of a patient. The methods and systems may provide an accurate and non-intrusive way for identifying strain history for a portion of a body of a patient through use of an efficient thin-film strain sensing material. In an embodiment, a thin-film sensor may be a composite of material. The composite may include an electrically resistant material, conductive nanoparticles dispersed throughout the electrically resistant material, and conductive nano-structures dispersed throughout the electrically resistant material. Silver nanoparticle based conductive adhesive or other appropriate materials may be used to make electrical leads or terminals that may connect the composite to circuit elements such as a voltage source. A gauge factor of the composite, which is a ratio of relative change in electrical resistance due to strain, may be greater than about 4 and may also vary with temperature, for example.

A thin-film sensor in accordance within an example (e.g., a CB/CNT/epoxy thin-film sensor) may be used for identifying a strain history for various portions of a body of a patient. The strain of a particular portion of a body of a patient may be due to various forces acting on the body, such as tensile forces, compressive forces, and torsional forces. A sensor apparatus may have a plurality of thin-film sensors that are arranged in a pattern suitable for detecting strain behavior for a given portion of a body of a patient. The plurality of thin-film sensors may be arranged in a wide variety of patterns, and thus, the thin-film sensor apparatus may serve to identify the strain behavior of any portion of a human body (or, more generally, any species). For example, the thin-film sensor apparatus may be used to detect the strain behavior in backs, joints, and other critical parts on a body of a patient. Example joints include but are not limited to an elbow joints, finger joints, toe joints, shoulder joints, hip joints, and knee joints. The thin-film sensor apparatus can be used non-intrusively because the sensor apparatus can be placed externally on the surface of the body (e.g., on a joint or on a back).

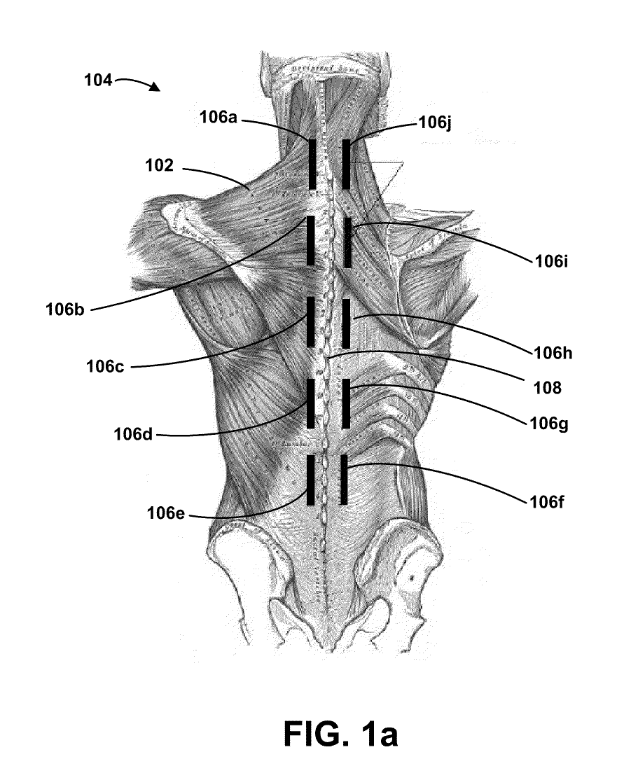

Referring now to the figures, FIG. 1(a) illustrates example thin-film sensors of an example sensor apparatus that may be applied to a portion of a body of a patient. FIG. 1(b) illustrates the sensor apparatus 100 that includes thin-film sensors 106a-j. Specifically, the sensor apparatus 100 can be applied to a back 102 of a patient 104. The sensors 106a-j are depicted as arranged in a pattern that includes two longitudinal arrays of sensors that are aligned up and down spine 108. These sensors may, for example, be disposed on a flexible membrane that is applied to the patient 108 using an adhesive. It should be understood that this depicted arrangement of the thin-film sensors is an example only. Other arrangements are possible. In addition, a sensor apparatus may include any suitable number of thin-film sensors. For example, any number of thin-film sensors from one or more may be used.

Further, the thin-film sensors may be various sizes. In an example, the thin film sensors 106a-j may be approximately 4 centimeters (cm)×1 cm. However, any dimension of the thin-film sensor may be used depending on an application and a total surface area available for mounting the sensor apparatus 100. For example, sensors of a larger dimension may be used on the human back than that can be used on and elbow or knee joint, as the back provides a larger surface area. In some cases, it may be ideal to monitor a large surface by using a pattern of the sensors. In such cases, sensors of smaller dimensions can be used to form a pattern or array and yet monitor the stress on larger areas.

Although not shown in FIG. 1(a), each thin-film sensor 106a-j may be connected to a first electrical lead and a second electrical lead. By being connected to two electrical leads, it is possible to apply a voltage across the thin-film sensor so that an electrical response of the thin-film sensor may be measured. In particular, electrical resistivity of the thin-film sensors 106a-j may be measured. The resistivity of the thin-film sensors 106a-j may vary predictably and measurably with a strain applied to the sensors, so that if the resistivity of a sensor is known, the magnitude of the strain applied to the sensor may be determined.

Returning to FIG. 1(a), the thin-film sensors 106a-j may be used to determine the strain history of back 102 and therefore forces to which the back 102 is subjected. The thin-film sensors 106a-j may detect, for example, bending, torsion, lateral forces, and longitudinal forces and strains exerted on or by back 102. The thin-film sensor strain history over a period of time may be accurately identified based on changes in the electrical response of the thin-film sensors 106a-j (e.g., changes in the resistance from a reference resistance).

In addition to thin-film sensors 106a-j, the sensor apparatus 100 may include additional elements. An example block diagram of sensor apparatus 100 is depicted in FIG. 1(b). As seen in FIG. 1(b), the sensor apparatus 100 may also include a processing unit 120 and a wireless communication interface 130. The thin-film sensors 106a-j may be in communication with processing unit 120. Processing unit 120 may operate according to an operating system, which may be any suitable commercially available embedded or disk-based operating system, or any proprietary operating system. Further, the processing unit 120 may comprise one or more smaller central processing units, including, for example, a programmable digital signal processing engine or may also be implemented as a single application specific integrated circuit (ASIC) to improve speed and to economize space. In general, it should be understood that the processing unit 120 could include hardware objects developed using integrated circuit development technologies, or yet via some other methods, or the combination of hardware and software objects that could be ordered, parameterized, and connected in a software environment to implement different functions described herein. Also, the hardware objects could communicate using electrical signals, with states of the signals representing different data.

The processing unit 120 is configured to measure an electrical response of the thin-film sensors 106a-j. In particular, the processing unit 120 may be configured to apply a voltage across a thin-film sensor and to detect a resulting electrical response of the sensor. As mentioned above, each thin-film sensor may be connected to a first electrical lead and a second electrical lead. These leads may be connected to the processing unit 120 and the processing unit 120 may control a voltage applied across the thin-film sensor. In an example, the processing unit 120 is also configured to allow for adjusting a bias voltage across the thin-film sensors. Therefore, the processing unit may include or be connected to a voltage source and a voltage amplifier, which may be used to apply different voltages across the thin-film sensors. Voltages supplied to the thin-film sensors 106a-j may be DC, AC, or DC and AC. Changing the biasing voltage may operate to tune the sensitivity of the thin-film sensors to a desired sensitivity.

Tuning the sensitivity may be useful in a variety of situations. In general, when deformation due to stretching, bending, or other forces is small, it may be useful to have a high sensitivity. For example, in limbs with straight fibrils of muscles, the deformation may be an expansive-type deformation or contractile-type deformation. In order to sense volume change by surface strain on the skin, it may be useful to have a high sensitivity for the thin-film sensors. A particular example is cough muscle. As another example of when high sensitivity may be desired, a small change in posture may lead to a small deformation (e.g., in the back muscles), and a high sensitivity may be useful in detecting these small changes. In general, high sensitivity may be useful for detecting small deformations in any part of the body.

The wireless communication interface 130 may be any wireless communication interface currently known in the art or later developed. In an example, wireless communication interface 130 may communicate with wireless communication interface 146 such that wireless communication interface 130 (i) sends data or instructions from example sensor apparatus 100 to data acquisition and analysis system 140 through wireless communication interface 146 and (ii) receives data or instructions from data acquisition and analysis system 140 through wireless communication interface 146. Wireless communication interface 130 may communicate with wireless communication interface 146 such that wireless communication interface 130 (i) sends data or instructions from data acquisition and analysis system 140 to example sensor apparatus 100 through wireless communication interface 130 and (ii) receives data or instructions from example sensor apparatus 100 through wireless communication interface 130.

The data acquisition and analysis system 140 may analyze the received data in order to identify the strain history behavior of back 102. The data acquisition and analysis system 140 may include, for example, a processor 142, memory 144, and a wireless communication interface 146. The processer 142 may process data received from the sensor apparatus 100. The processor 142 may be embodied as a processor that accesses the memory 144 to execute software functions stored therein. Processor 142 may be similar to processor 120.

The memory 144 may store information such as previously transmitted or received data from the sensor apparatus 100, for example. The memory 144 may include random access memory (RAM), flash memory or long term storage, such as read only memory (ROM) or magnetic disks, for example. Further, wireless interface 146 is configured to communicate with the corresponding wireless communication interface 130 of sensor apparatus 100.

In an example, data acquisition and analysis system 140 may also include or be in communication with a band pass filter and an oscilloscope. The band pass filter and oscilloscope may facilitate the analysis of the data received from processing unit 120 via wireless interface 130. The band pass filter may be used to separate the actual sensor signal from the background unwanted signal generally referred to as noise. Noise may comprise of stray vibrations in the surroundings, signal generated due to blood flow in the body, respiration, cardiac activity, and so forth. In a band pass filter, signals of undesired frequencies may be truncated from the output signal. Further, an oscilloscope is a display device that may be used to observe the signal.

A sensor apparatus, such as sensor apparatus 100, may be applied to a patient in a variety of ways. In an example, the thin-film sensors 106a-j of the sensor apparatus may be disposed on a flexible membrane that may then be attached to the patient with an adhesive. The flexible membrane may be a membrane that is capable of flexing in any dimension, and thus may be applied to any surface. Example flexible-membrane materials include polycarbonate, latex, Teflon, and silicon rubber. In general, any flexible, thin polymer, or fabric may be used.

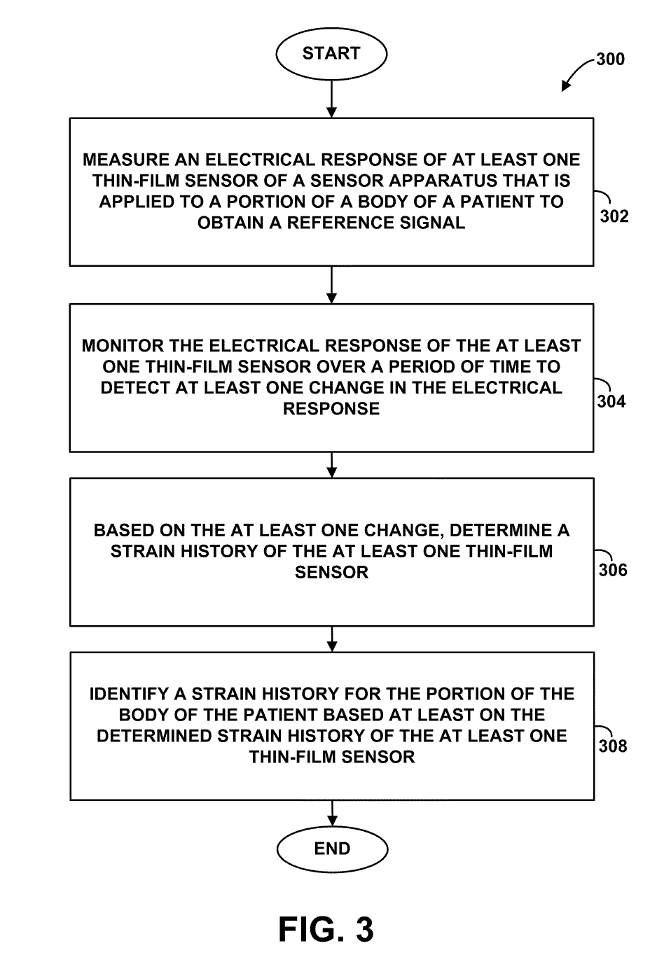

In addition, all of the thin-film sensors of a sensor apparatus may be disposed on a single flexible membrane. For example, for a sensor apparatus intended for identifying the strain history of a knee-cap, all the thin-film sensors may be disposed on a flexible membrane about the size of the knee-cap. In another example, however, the sensor apparatus may include multiple flexible membranes having thin-film sensors disposed. For instance, in the case of FIG. 1(a) where the sensor apparatus is intended to be applied to the back, multiple flexible membranes may be used. For example, each one of sensors 106a-j may be disposed on its own flexible membrane. Other examples are possible as well. The flexible membrane may be applied to a patient's clothing, such as the patient's socks or gloves. In general, a flexible membrane may be applied on any accessory the patient may wear. For round parts of the body (e.g., a wrist or an ankle), the thin-film sensors may be applied to an elastic band that is configured to fit around the body part. In an alternative example, the thin-film sensor may be directly applied to the patient. Example configurations of sensor apparatuses are shown in FIG. 4 described below.

As discussed above, in the example of FIGS. 1(a)-(b), the sensor apparatus 100 and the data acquisition and analysis system 140 wirelessly communicate with one another. Therefore, in this example, the patient 104 would not need to transport the data acquisition and analysis system 140 when the sensor apparatus 100 is applied to the patient 104. However, it should be understood that in an example, the sensor apparatus may itself include a data acquisition and analysis system.

The electrically resistant material of the composite of a thin-film sensor, such as thin-film sensor 106a, may be an electrical insulator, or other material with relatively low conductivity (and relatively high resistivity). The electrically resistant material may be a polymer, such as epoxy resin. The electrically resistant material may also be a matrix in which the nanoparticles and the nano-structures are dispersed, suspended, or embedded, for example.

The conductive nanoparticles may constitute approximately 33% of a total volume of the composite. The conductive nanoparticles may be amorphous carbon, such as carbon black (CB). The conductive nanoparticles may also be platinum, silver, copper, and polyanylene. The conductive nanoparticles may also be a conductive polymer backbone and nanowires made of polyanylene and conjugate polymers, for example. A concentration of nanoparticles like CB provides a permanent conducting pathway in the composite (the existence of the conducting pathway not being dependent upon a strain experienced by the composite), which increases reliability and repeatability of measurements made using the composite. The addition of nanoparticles like carbon black may make the composite sufficiently conductive (by reducing electrical impedance) so that minimal electrical power is consumed. In an embodiment in which a thin-film sensor, such as thin-film sensor 106a, includes carbon black and epoxy, the carbon black may lower the resistance of the epoxy from mega ohms to a range of a few kilo ohms, for example.

The conductive nano-structures may be carbon nanotubes (CNT), and in one example may constitute less than about 5% of the total weight of the composite. In another example, the conductive nano-structures may also constitute less than about 1% of the total weight of the composite. The CNTs used as the conductive nano-structures may be highly pure grade or may have metallic particles or carbon particle impurities. The CNTs may be more than about 10 micrometers long, and may have diameters in a range of less than about a nano-meter to hundreds of nanometer.

The thin-film sensor 106a may be prepared by processing carbon black nanoparticles and carbon nanotubes in epoxy resin by electromechanical and mechanochemical methods. As one example, the dispersion technique of ultrasonification may be used to disperse the CNTs and to increase the gauge factor of the composite. As another example, spin coating may be used to coat CNTs on the surface of the epoxy resin or the surface of an epoxy and carbon black composite. As yet another example, with typical dispersion techniques, CNTs may be randomly aligned with respect to each other. As yet an additional example, to align the CNTs, a deep-coating method with a slow draw out of a deep-coating solvent may be used. The thin-film sensor 106a may be made in any desired shape, pattern, or area. The thin-film sensor 106a may also be flexible and applied on irregular and stretchable surfaces using standard adhesives for bonding.

A residual strain or residual stress may arise in thin film 106a if, at the time of manufacturing, a base substrate or a mould is not stress free. For example, the base substrate may be bent or deformed when in a green viscous state or may be poured into a deformed mould. Therefore, after polymerization, residual stress arising out of the deformation of the substrate or the mould may remain present in thin film sensor 106a. Temperature-induced shrinkage may also cause stress in thin film sensor 106a and may occur because of polymerization at an elevated temperature and subsequent cooling. The amount of residual stress in thin film sensor 106a may be determined by comparing electric and electro-mechanical properties with the electric and electro-mechanical properties of a stress-free sensor.

A change in the resistance of epoxy, carbon black, and carbon nanotube of the thin-film sensor 106a due to an applied uniaxial stress may be attributed to a change in a volume fraction of non-conducting epoxy of the thin-film sensor 106a. Because an elastic moduli of the epoxy matrix differs from those of the carbon black and carbon nanotube fillers, the epoxy deforms more than the carbon black particles and carbon nanotubes under stress or strain. This difference in degree of deformation leads to a change in an effective energy band-gap in the thin-film sensor 106a. As a result, resistance of the thin-film sensor 106a changes as a function of applied stress.

In example embodiments of thin film sensor 106a including epoxy, carbon black nanoparticles, and carbon nanotubes, operation and properties of thin film sensor 106a may be explained using the following parameters:

L₀ is an initial length of the thin-film sensor 106a.

A₀ is an initial Area of cross section of the thin-film sensor 106a.

εₓₓ is an uniaxial tensile strain in the thin-film sensor 106a.

V₀ is an initial volume of the thin-film sensor 106a.

Vₘ is an initial volume of epoxy in the thin-film sensor 106a.

V_{cb} is an initial volume of carbon black in the thin-film sensor 106a.

V_{cnt} is an initial volume of CNT in the thin-film sensor 106a.

Vₘ^{new} is a new volume of epoxy due to an application of εₓₓ.

fₘ is an initial volume fraction of epoxy in the thin-film sensor 106a.

fₙ^{new} is a new volume fraction of epoxy due to an application of εₓₓ.

ε^{(m)} is a strain in an epoxy phase.

σ is the stress.

E_{eff} is the Young's modulus of carbon black/epoxy thin-film sensor 106a.

Eₘ is the Young's modulus of epoxy.

E_{cb} is the Young's modulus of carbon black.

νₘ is the Poisson's ratio of epoxy.

ν is the Poisson's ratio of carbon black.

φ is an applied bias voltage.

ψ is an average orientation of carbon chains with respect to applied electric field.

f_{cb} is a volume fraction of carbon black in the thin-film sensor 106a.

κₘ is an electrical conductivity of epoxy.

κ_{cb} is an electrical conductivity of carbon black.

κₑ is an effective electric conductivity of carbon black/epoxy thin-film sensor 106a.

E₀ is an effective electric field.

κ_{eff} is an effective electrical conductivity of CNT/carbon black/epoxy thin-film sensor 106a

κ_{S} is an electrical conductivity of an interfacial layer.

κ_{cnt} is an electrical conductivity of a single CNT.

κ_{com}⁽¹¹⁾ is a transverse electrical conductivity of a complex CNT.

κ_{com}⁽³³⁾ is a longitudinal electrical conductivity of a complex CNT.

j is a spatially varying electrical current density.

L₀ and A₀ (A₀<<L₀²) may be the initial length and initial cross-sectional area of the film on which the uniaxial stress is applied. A volumetric change in the epoxy matrix resulting from applied uniaxial stress in the thin-film sensor 106a is present, but any volumetric change specifically in the carbon black and carbon nanotube fillers is negligible. Because any volumetric change is experienced primarily in the epoxy matrix and not in the carbon black nanoparticles and the carbon nanotubes, the relative volumes of epoxy, carbon black, and carbon nanotubes changes when strain is applied to thin film sensor 106a. For a uniaxial strain εₓₓ applied to the thin-film sensor 106a with total initial volume V₀, initial volume of epoxy Vₘ, volume of carbon black filler V_{cb} and volume of CNT V_{cnt}, a new volume V of the thin-film sensor 106a in terms of the new volume of epoxy, Vₘ^{new}, may be given by Equation 1:

V=Vₘ^{new}+V_{cb}+V_{cnt}=V₀(1+εₓₓ) Equation (1)

fₘ is an initial volume fraction of epoxy in the thin-film sensor 106a, and a new volume fraction fₘ^{new} may be given by Equation 2:

$${f}_{m}^{new} = \frac{{V}_{m}^{new}}{V} = \frac{{V}_{m}^{new}}{{V}_{0} ( 1 + {ɛ}_{xx} )} \qquad Equation ( 2 )$$

Vₘ^{new} may also be written in terms of strain developed in the epoxy as Vₘ^{new}=Vₘ(1+ε^{(m)}), where ε^{(m)} is a strain developed in the epoxy and Vₘ=fₘ^{V}₀. This yields Equation 3:

$${f}_{m}^{new} = {f}_{m} [ \frac{1 + {ɛ}^{( m )}}{1 + {ɛ}_{xx}} ] \qquad Equation ( 3 )$$

ε^{(m)} is greater than εₓₓ as a stiffness of epoxy is less than that of the carbon black nanoparticle reinforced thin-film sensor 106a. Hence, Equation 3 shows that a volume fraction of epoxy increases with the applied strain. As resistivity of epoxy is greater than that of the thin-film sensor 106a, a resistance of the thin-film sensor 106a also increases with an increase in a volume fraction of epoxy. εₓₓ is computed from an effective stiffness of the thin-film sensor 106a E_{eff} whereas ε^{(m)} is nonlinear and is computed from a phenomenological constitutive model of glassy polymers, for example.

The strain ε^{(m)} in the polymer (epoxy) can be first written in a rate form and the phenomenology of pre-yield softening is adopted. Mechanical properties of polymers are dependent not only on a strain applied on them, but also on a time rate of application of strain. Therefore, the mechanical response of a polymer on which strain is applied at one rate would be different than the mechanical response of a polymer on which strain is applied at a higher rate. Hence, the rate effect may be included in a strain calculation. A yield point is a point on a stress-strain curve when the material tend to become inelastic (i.e., elastic recovery is not possible beyond this point of stress-strain state). Softening means a decrease in a slope of the stress-strain curve. A pre-yield softening is observed in most polymers where a non-linear behavior is observed in the stress-strain relationship owing to fluctuation in the elastic properties of the polymers. Polymers generally soften prior to yield when strained sufficiently. Hence, pre-yield softening may be included in a strain calculation.

E_{eff} is the effective Young's modulus of a two component (CB/epoxy) used as a medium for CNT inclusion to create thin-film sensor 106a. This depends on the Young's moduli of the components, Eₘ for epoxy and E_{cb} for carbon black, the volume fraction of each component, fₘ for epoxy and f_{cb}, for carbon black, respective Poisson's ratios (σₘ and σ_{cb}), applied bias voltage φ, average angle of orientation ψ of the carbon chains with respect to the electric field, and the number of carbon atoms in the carbon chains. For a thin-film sensor 106a with volume fraction f_{cb}, a small number of new particles may be theoretically added. An increment in Young's modulus dE_{eff} resulting from an addition of the new particles may be calculated from a dilute system result by treating the thin-film sensor 106a to which the new particles are added as an equivalent effective medium of Young's modulus E_{eff} according to Equation 4:

dE_{eff}=E_{eff}Kdf_{cb}+E_{eff} f_{cb}dK Equation(4)

which expands into Equation 5:

$$\frac{d {E}_{eff}}{d {f}_{eb}} = [ \frac{{E}_{ef} f ( {f}_{cb}^{2} {N}_{1} + {f}_{cb} ( {N}_{2} {E}_{eff} + {N}_{3} ) )}{( \begin{matrix}{f}_{cb}^{2} {M}_{1} + {f}_{cb} ( {M}_{2} {E}_{eff} + {M}_{3} ) + \\ ( {M}_{4} {E}_{eff}^{2} + {M}_{5} {E}_{eff} + {M}_{6} )\end{matrix} )} ] + [ \frac{{E}_{eff} ( {N}_{4} {E}_{eff}^{2} + {N}_{5} {E}_{eff} + {N}_{6} )}{( \begin{matrix}{f}_{cb}^{2} {M}_{1} + {f}_{cb} ( {M}_{2} {E}_{eff} + {M}_{3} ) + \\ ( {M}_{4} {E}_{eff}^{2} + {M}_{5} {E}_{eff} + {M}_{6} )\end{matrix} )} ] + [ \frac{\begin{matrix}{E}_{eff} {f}_{cb} ( 2 {f}_{cb} {N}_{1} + ( {N}_{2} {E}_{eff} + {N}_{3} ) ) \\ ( {f}_{cb}^{2} {M}_{1} + {f}_{cb} ( {M}_{2} {E}_{eff} + {M}_{3} ) )\end{matrix}}{{( \begin{matrix}{f}_{cb}^{2} {M}_{1} + {f}_{cb} ( {M}_{2} {E}_{eff} + {M}_{3} ) + \\ ( {M}_{4} {E}_{eff}^{2} + {M}_{5} {E}_{eff} + {M}_{6} )\end{matrix} )}^{2}} ] + [ \frac{\begin{matrix}{E}_{eff} {f}_{cb} ( 2 {f}_{cb} {N}_{1} + ( {N}_{2} {E}_{eff} + {N}_{3} ) ) \\ ( {M}_{4} {E}_{eff}^{2} + {M}_{5} {E}_{eff} + {M}_{6} )\end{matrix}}{{( \begin{matrix}{f}_{cb}^{2} {M}_{1} + {f}_{cb} ( {M}_{2} {E}_{eff} + {M}_{3} ) + \\ ( {M}_{4} {E}_{eff}^{2} + {M}_{5} {E}_{eff} + {M}_{6} )\end{matrix} )}^{2}} ] + [ \frac{\begin{matrix}{E}_{eff} {f}_{cb} ( 2 {f}_{cb} {M}_{1} + ( {M}_{2} {E}_{eff} + {M}_{3} ) ) \\ ( {f}_{cb}^{2} {N}_{1} + {f}_{cb} ( {N}_{2} {E}_{eff} + {N}_{3} ) )\end{matrix}}{{( \begin{matrix}{f}_{cb}^{2} {M}_{1} + {f}_{cb} ( {M}_{2} {E}_{eff} + {M}_{3} ) + \\ ( {M}_{4} {E}_{eff}^{2} + {M}_{5} {E}_{eff} + {M}_{6} )\end{matrix} )}^{2}} ] + [ \frac{\begin{matrix}{E}_{ef} {f}_{cb} ( 2 {f}_{cb} {M}_{1} + ( {M}_{2} {E}_{eff} + {M}_{3} ) ) \\ ( {N}_{4} {E}_{eff}^{2} + {N}_{5} {E}_{eff} + {N}_{6} )\end{matrix}}{{( \begin{matrix}{f}_{cb}^{2} {M}_{1} + {f}_{cb} ( {M}_{2} {E}_{eff} + {M}_{3} ) + \\ ( {M}_{4} {E}_{eff}^{2} + {M}_{5} {E}_{eff} + {M}_{6} )\end{matrix} )}^{2}} ] \qquad Equation ( 5 )$$

where Mᵢ and Nᵢ are constants and given by Equation 6:

M₁=A₅B₄, M₂=2α₆A₅+α₄B₄

M₃=A₄B₄+A₅E_{c0} M₄=2α₄α₆

M₅=2Aα₄+α₄E_{c0} M₆=A₄E_{c0}

N₁=C₁A₂B₄+C₂B₂A₅

N₂=2α₆C₁A₂−C₁α₂B₄+C₂B₂α₄−C₂A₅α₂α₅

N₃=C₁A₁B₄+C₁A₂E_{cO}+C₂B₁A₅+C₂B₂A₄

N₄=−(2C₁α₂α₆+C₂α₂α₄α₅)

N₅=2C₁A₁α₆−C₁α₂E_{c0}+C₂B₁α₄−C₂A₄α₂α₅

N₆=C₁A₁E_{cO}+C₂B₁A₄ Equation (6)

The effective Young's modulus of the composite E_{eff} may be computed according to Equation 4 by integrating Equation 4 numerically using a fourth order Runge-Kutta scheme.

The effective electrical conductivity κₑ of the CB/epoxy background of thin-film sensor 106a, which is a function of the volume fractions of the constituents, may be computed next using the effective medium approximation (EMA) considering both components as randomly dispersed isotropic spherical grains. The relative volume fraction of epoxy and carbon black may be fₘ and f_{cb}, respectively, where f_{cb}=(1−fₘ), and the epoxy and carbon black may have conductivities κₘ and κ_{cb}, respectively. According to the EMA, each component grain is considered to be immersed in a homogeneous effective medium of conductivity κₑ instead of being embedded in its actual random background environment. Equation 7:

$${f}_{m} [ \frac{{κ}_{m} - {κ}_{e}}{{κ}_{m} + 2 {κ}_{e}} ] + ( 1 - {f}_{m} ) [ \frac{{κ}_{cb} - {κ}_{e}}{{κ}_{cb} + 2 {κ}_{e}} ] = 0 \qquad Equation ( 7 )$$

gives an effective electrical conductivity of the unstrained CB/epoxy background of thin-film sensor 106a. Substituting the strain-dependent volume fraction of epoxy fₘ^{new}=fₘ^{new}(σ, ε) in place of fₘ yields Equation 8:

$${f}_{m}^{new} [ \frac{{κ}_{m} - {κ}_{e}}{{κ}_{m} + 2 {κ}_{e}} ] + ( 1 - f \frac{new}{m} ) [ \frac{{κ}_{cb} - {κ}_{e}}{{κ}_{cb} + 2 {κ}_{e}} ] = 0 \\ ⇒ [ {f}_{m} ( \frac{1 + {ɛ}^{( m )}}{1 + {ɛ}_{xx}} ) ] [ \frac{{κ}_{m} - {κ}_{e}}{{κ}_{m} + 2 {κ}_{e}} ] + [ 1 - {f}_{m} ( \frac{1 + {ɛ}^{( m )}}{1 + {ɛ}_{xx}} ) ] [ \frac{{κ}_{cb} - {κ}_{e}}{{κ}_{cb} + 2 {κ}_{e}} ] = 0 , \qquad Equation ( 8 )$$

where ε^{(m)} is computed as above.

The effective electrical conductivity κ_{eff} of the three component thin-film sensor 106a (CB/CNT/epoxy) may be computed considering the background κₑ to which CNTs are added. κₑ is a conductivity of the base matrix, and CNTs are considered to be randomly dispersed prolate ellipsoidal inclusions (all of the same shape) in this matrix, for example. For high aspect ratios, a cylinder can be suitably modeled by a prolate spheroid without introducing appreciable errors into a final solution. Since the effective fiber retains geometrical dimensions of the nanotube, the effective fiber's aspect ratio will also be high, and thus may be modeled with a prolate spheroidal inclusion. In modeling the cylindrical geometry with a spheroid, aspect ratios are substantially equivalent, meaning that a volume of the spheroidal inclusion will not be the same as that of the cylindrical inclusion. However, due to small dimensions of the multiwalled carbon nanotubes, this difference in volume may not significantly affect a volume fraction of the inclusion phase. The conductivity may be calculated using a generalized EMA model incorporating the interface shell effect.

A CNT may be coated with a thin interfacial layer of conductivity κ_{S}, and the CNT as a whole can be considered as a complex CNT. A quantum effect may also be regarded as a kind of interfacial effect, which affects the electrical conductivity of the composite accordingly. κ_{cnt}, κₑ, κ_{S} and κ_{eff} are the electrical conductivities of the CNT, matrix, interfacial layer and final composite.

The effective electrical conductivity κ_{eff} of the complex CNTs and the (epoxy/CB) matrix may be calculated using a generalized EMA modeling the complex CNTs as prolate ellipsoids randomly mixed with spherical matrix particles. The effective electrical conductivity κ_{eff} of the thin-film sensor 106a is defined as [symbol] \vec{j} [symbol] =κ_{eff}\vec{E}₀, where [symbol] \vec{j} [symbol] is the volume average of the spatially varying current density. \vec{E}₀ is the volume average of \vec{E}, i.e. \vec{E}= [symbol] \vec{E} [symbol] . B_{com,k} and B_{m,k} are depolarization factors of the complex CNTs and matrix particles, f_{cnt} is a volume fraction of CNTs in the composite, α is a ratio of a volume of the CNT and a volume of a complex CNT. The depolarization factors for spherical particles are taken as B_{m,x}=B_{m,y}=B_{m,z}=⅓, and that for the prolate ellipsoids, assuming L/(2R)>>1, B_{com,x}=B_{com,y}=(1−B_{com,z})/2. Given these values, and taking the final composite to be effectively isotropic, yields Equation 9:

$$\frac{{f}_{cnt}}{3 α} [ \begin{matrix}\frac{{κ}_{eff} - {κ}_{com}^{( 33 )}}{{κ}_{eff} + {B}_{com , z} ( {κ}_{com}^{( 33 )} - {κ}_{eff} )} + \\ 4 \frac{{κ}_{eff} - {κ}_{com}^{( 11 )}}{2 {κ}_{eff} + ( 1 - {B}_{com , z} ) ( {κ}_{com}^{( 11 )} - {κ}_{eff} )}\end{matrix} ] + 3 ( 1 - \frac{{f}_{cnt}}{α} ) \frac{{κ}_{eff} - {κ}_{e}}{2 {κ}_{eff} + {κ}_{e}} = 0 \qquad Equation ( 9 )$$

Equation 9 can be solved for κ_{eff} with a value for the background effective conductivity of the CB/epoxy matrix from Equation 8. f_{cnt} may be written as a function of the applied strain according to Equation 10:

$${f}_{cnt}^{new} = {f}_{cnt} [ \frac{1}{1 + {ɛ}_{xx}} ] \qquad Equation ( 10 )$$

where f_{cnt}^{new} is a new volume fraction of the CNTs in the composite due to the applied strain εₓₓ. ε^{(cnt)} is the strain developed in the CNTs, which can be obtained from the Young's Modulus of the CNT. Given that the strain-dependent volume fraction of CNT may be expressed by f_{cnt}^{new}=f_{cnt}^{new}(σₓₓ, εₓₓ), Equation 11:

$$( \frac{{f}_{cnt}}{3 α} ) ( \frac{1 + {ɛ}^{cnt}}{1 + {ɛ}_{xx}} ) [ \begin{matrix}\frac{{κ}_{eff} - {κ}_{com}^{( 33 )}}{{κ}_{eff} + {B}_{com , z} ( {κ}_{com}^{( 33 )} - {κ}_{eff} )} + \\ 4 \frac{{κ}_{eff} - {κ}_{com}^{( 11 )}}{2 {κ}_{eff} + ( 1 + {B}_{com , z} ) ( {κ}_{com}^{( 11 )} - {κ}_{eff} )}\end{matrix} ] + 3 [ 1 - \frac{{f}_{cnt}}{α} ( \frac{1 + {ɛ}^{( cnt )}}{1 + {ɛ}_{xx}} ) ] \frac{{κ}_{eff} - {κ}_{e}}{2 {κ}_{eff} + {κ}_{e}} = 0 \qquad Equation ( 11 )$$

gives a relationship between the applied strain, εₓₓ, and the effective conductivity, κ_{eff}, of the CB/CNT/epoxy composite. κ_{eff} may be calculated using the value of κₑ obtained from Equation 8.

The foregoing is one description of properties and operation of a carbon black, carbon nanotube, and epoxy embodiment of thin-film sensor 106a; however other descriptions of the properties and operation of such a composite, and other strain sensing composites, are possible.

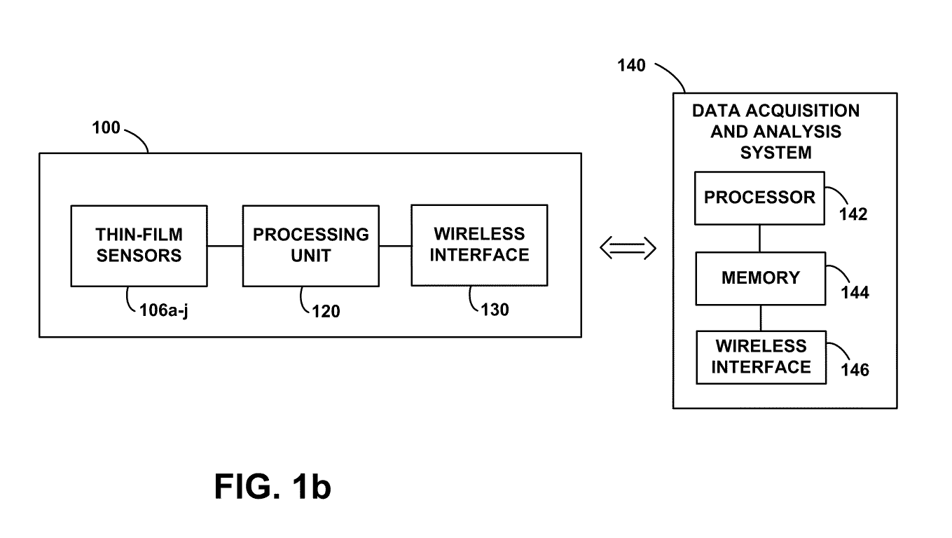

As mentioned above, each thin-film sensor of sensor apparatus 100 may measure the strain induced on the back 102 of patient 104 by various forces. FIG. 2 shows the sensor 106a that is disposed on the back 102. Further, FIG. 2 shows example forces 202 and 204 to which the back 102 may be subjected. These forces may induce strain in the thin-film sensors, which may then cause a change in the electrical response of thin-film sensor 106a monitored by sensor apparatus 100. The data related to the change in electrical response may be sent to data acquisition and analysis system 140 for identification of a strain history of spine 108.

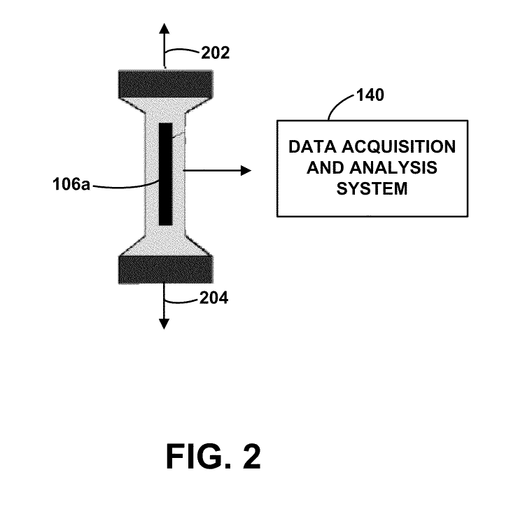

Identifying the strain behavior of a portion of a body of a patient, such as spine 108 of patient 104, is described in further detail with reference to FIG. 3. FIG. 3 is a flowchart that depicts example steps of a method for identifying stress behavior of a portion of a body of a patient. It should be understood that the flowchart shows functionality and operation of one possible implementation of present embodiments. Method 300 begins at block 302 where an electrical response of at least one thin-film sensor that is applied to a portion of a body of a patient is measured to obtain a reference signal. Then at block 304, the electrical response of each of the at least one thin-film sensor over a period of time is monitored to detect at least one change from the reference signal in the electrical response. Next, at block 306 a strain history of the at least one thin-film sensor is determined based on the at least one change from the reference signal in the electrical response. Finally, at block 308, a strain history for the portion of the body of the patient is identified based at least on the determined strain history of the at least one thin-film sensor.

After the sensor apparatus 100 is attached to patient 104, at block 302, an electrical response of each thin-film sensor in the sensor apparatus (i.e., sensors 106a-j) may be measured to obtain a reference signal. Processing unit 120 may measure this electrical response by applying a voltage across the thin-film sensor and detecting the electrical response. This measurement will give a “base-line” or “reference signal” that may serve as a basis for monitoring the electrical response of the thin-film sensor over a period of time to detect changes in the electrical response.

In accordance with an example, the reference signal may be the signal obtained when the sensor apparatus is under a no load condition. As an example, a no load condition for patient 104 may be when the patient's spine 108 is straight and the patient is standing. Other example no load conditions include a straight elbow, wrist, back muscles, and spine when a patient is in a straight sitting posture. Yet another example of a no load condition is a straight knee joint when a patient is lying down. In a sitting apparatus or a man-machine interface, where a patterned thin-film sensor is being used to monitor ergonomic compatibility or overstress of the patient, a non-load condition may imply that the apparatus or interface is under free, unattended, or unused conditions.

The reference signal of a thin-film sensor of a sensor apparatus will likely be different depending of a surface to which the sensor apparatus is applied. When the stretchable and flexible membrane with a printed thin-film sensor or thin-film sensors is placed over a large irregularity, the printed sensor or sensors would give a reference signal. Due to the irregular surface, the thin film sensor or sensors will experience stretching at the base-line condition, and hence will change electrical conductivity due to the inherent sensitivity of the film material. Subsequent signals for joint property can be made with reference to this reference signal. Thus, the reference signals of thin-film sensors applied to different subjects and different joints will likely be different. For example, a reference signal for a first thin-film sensor disposed on a first knee-cap of a first patient will likely be different than a reference signal for a second thin-film sensor disposed on a second knee-cap of a second patient. This is true because the surface of each respective knee cap will likely be different, and thus the sensors will bend differently when applied to the respective knee caps.

After the reference signal is measured for each thin-film sensor 106a j, sensor apparatus 100 monitors the electrical response of each thin-film sensor 106a-j to detect at least one change from the reference signal in the electrical response. As the portion of the body of the patient is subject to stress, the portion of the body will experience strain. This strain will cause the thin-film sensors to likewise experience strain, and the induced strain will change the electrical response of the thin-film sensors. The processing unit 120 may monitor these changes and may send the electrical response changes to data acquisition and analysis system 140. Alternatively, processing unit 120 may send the raw electrical response data to data acquisition and analysis system 140. The data acquisition and analysis system 140 may then detect these changes in the electrical response of the thin-film sensors.

Progressive monitoring of the electrical response of the thin-film sensor can be accomplished by measuring the electrical response signals from the individual thin-film sensors over a period of time. In an example, monitoring the electrical response of the thin-film sensor may take place in real time or in substantially real time. Alternatively, the processing unit 120 may monitor the electrical resistance by measuring the electrical resistance of the thin-films periodically (e.g., every 5-10 milliseconds, every 1 second, every 5 seconds, etc).

At block 306, after at least one change in the electrical response of the thin-film sensor is detected, the data analysis system 140 determines a strain history of the thin-film sensor. As discussed above, the electrical response of the thin-film sensors changes when stress is applied to the sensor. Accordingly, a particular strain may be correlated with a given electrical response of the thin-film sensor. Data analysis system 140 may use the electrical response measurements to determine the magnitude of strain applied to the thin-film sensor. In an example, data analysis system may use the measurements to solve the equations to determine the applied strain. Conductivity, κ, and resistance, R, are related as in Equation 12:

$$R = \frac{L}{κ A} \qquad Equation ( 12 )$$

Equation 11 expresses the relationship between the applied strain (εₓₓ) and the conductivity of the sensor (κ_{eff}). Data analysis system 140 may take the measured resistance and may use that measured resistance to determine conductivity, κ_{eff}, using Equation 12. Data analysis system 140 may then solve Equation 11, using the determined conductivity as an input, to determine the applied strain, εₓₓ. In Equation 11, all terms except κ_{eff}, εₓₓ and κₑ may be constants, and κₑ may be obtained from Equation 8. Alternately, data analysis system 140 may have access to look-up tables that map values or ranges of values measured by the thin-film sensor onto values or ranges of values of applied strain. Such a look-up table may be generated when the thin-film sensor is designed, constructed, or calibrated. Data analysis system 140 may use the determined value or values of the applied strain in other calculations.

Additional factors may be taken into account when determining the strain history of the thin-film sensor. For instance, a bias voltage may be applied across the thin-film sensor, in which case the determination of the applied strain would take into account the applied bias voltage. Also, a temperature of the flexible thin film strain sensor may be measured, and the determination of the applied strain may take into account the temperature. As another example, a residual strain or stress bearing on the thin-film sensor may be ascertained, in which case the determination of the applied strain would take into account the residual strain or stress. Residual strain may be present because of deformations of a substrate or a mould during a manufacturing process; and residual strain may be ascertained by comparing sensor 106a to a strain-free sensor.

To obtain a strain history for each thin-film sensor, data analysis system 140 may determine a magnitude of strain of each thin-film sensor at various points in time. For example, over the course of a given time period, the data analysis system may calculate the strain of the thin-film sensor every second. It should be understood, however, that the system may determine the strain more often or less often (e.g., every 1 millisecond or every 30 seconds). As discussed above, the magnitude of the strain may be determined based on the difference between the electrical response at the point in time and the reference signal. The analysis system 140 may then compile the determined magnitudes of the strain of the thin-film sensor at the points in time to represent the strain history of the thin-film sensor. In an example, the analysis system 140 may compile the magnitude in a graph that plots strain magnitude against time.

After the strain history for each thin-film sensor of sensor apparatus 100 is determined, a strain history for the spine 108 is identified based at least on the determined strain history for sensors 106a-j at step 308. The strain history for spine 108 may take into account the strain determined for each thin-film sensor. Based on the strains of the individual sensors, an overall strain behavior of spine 108 may be determined.

In accordance with one example, the sensor apparatus may be used to determine whether the portion of the body the sensor apparatus is applied to suffers from any sort of medical issue. For instance, the sensor apparatus may be used to facilitate the determination of whether the patient may suffer from a joint ailment, such as joint deterioration, arthritis, osteoporosis, plantar fasciitis, osteomalacia, or rickets. To detect a joint ailment, the strain history of a patient's joint may be compared to a predetermined strain history. This predetermined strain history may be a predetermined strain history of a healthy or ideal joint.

In an example, the predetermined strain history is determined by identifying a strain history of test subject by using by a sensor apparatus substantially similar to the sensor apparatus used for the subject patient. That is, the sensor apparatus used on the test subject is substantially similar in design to the one used for the patient (e.g., similar number and pattern of thin-film sensors). The test subject may be a real human subject with healthy joints or a lab-scale specimen designed to simulate a healthy subject. Using this type of technique, it is possible to map the strain history for a wide variety of joints. Differences in the strain history of a subject patient's joint and strain history of a test subject (e.g., with a healthy or ideal joint) may provide an indication of a joint ailment.

What is considered a “healthy joint” or “ideal joint” may depend on various factors, such as age, weight, and height. For example, a healthy or ideal joint for a ten year old child may be different than a healthy or ideal joint for a 30 year old adult. As another example, a healthy or ideal joint for someone that is five feet tall may be different than a healthy or ideal joint for someone that is six feet tall.

In example embodiments, a sensor apparatus may be any suitable shape and size. For example, a sensor apparatus designed for a human back may be different that a sensor apparatus designed for a knee cap. The sensor arrangement may be printed on any shape and size of a flexible membrane. In an example, for bone/joint detection, a grid of one-dimensional channels of the thin-film can be printed on a stretchable membrane (e.g., a knee-cap). The orientations of these printed channels are preferably such that they experience strain due to various modes of joint motion and also load-transfer directly. Example load transfers include load transfers due to body weight under standing conditions, walking motion, etc.

In addition, thin films of any pattern of sensors may be constructed. The thin-film sensors of a sensor arrangement may be arranged in a pattern suitable for detecting stress behavior for the intended portion of the body of the patient. The pattern of the thin-film sensors may be selected based on various factors. For example, the pattern of the thin film may depend on the type of force or forces or strain to be measured (e.g., tensile, compressive, shear, etc.). As another example, the pattern of the thin-film sensors may depend on the amount of surface area to be covered by the sensor. As yet another example, the pattern of the thin-film sensors may depend on the number of points on which measurements need to be done. As still yet another example, the pattern of the thin-film sensors may depend on the topology of the surface. Other factors that affect the preferred pattern of the thin film are possible as well.

After the pattern of the thin-film sensors is ascertained based on the aforementioned factors, sensors can be arranged to form the desired pattern in order to measure the forces and strain both accurately and efficiently. In general, the sensors may be used in any pattern or array, such as a circular, rectangular, or 3-dimensional array to measure strains over a large area or volume both accurately and efficiently. As a particular example, a circular pattern may be useful in measuring the strains in joints such as the knee, the elbow, and the wrist. As another particular example, a rectangular or straight array may be useful in measuring strains in back muscle and the spine.

The thin-film sensors may also be useful for sensing ergonomic performance of sitting apparatuses and other human support accessories. In such cases, a thin-film pattern may be designed such that the distribution of pressure exerted by a human body can be sensed as a reference or benchmark. In another example, undesired pressure distribution that may lead to, for example, thrombosis may be captured by a suitable patterned array of thin-film sensors on the sitting apparatus or man-machine interface.

FIGS. 4(a)-(b) and 4(c)-(d) depict two example possible patterns of example thin-film sensors. As will be discussed below, these two example patterns are intended to measure different types of forces. A first example pattern 400 is shown in FIGS. 4(a)-(b). Specifically, FIG. 4(a) shows a pattern 400 and FIG. 4(b) shows a schematic representation of pattern 400, with a close-up of one of the node's of pattern 400. This example pattern may be designed to measure forces arising due to impact loading of an aluminum plate. In pattern 400, there are 25 nodes 402, and as seen, each of these 25 nodes (such as node 402) form a grid-like pattern. Specifically, this example forms a square grid. Each node 402 has two sensors 404a-b arranged in a “+” formation to measure the orthogonal strains at the 25 locations. However, sensors in each node may be arranged in different configurations, such as an “X” formation. A grid-like pattern as depicted in FIGS. 4(a)-(b) may be useful for measuring stress behavior on any part of the body where the available surface area for measurement is large and more than one type of force acts on the area. For example, a grid-like pattern may be used on the human back. Other examples where a grid-like pattern may be used include the thigh region, the chest region, and the stomach region. Similar to thin-film sensors 106a-j, sensors 404a-b may be thin-film sensors including an electrically resistant material, conductive nanoparticles dispersed substantially throughout the electrically resistant material, and conductive nano-structures dispersed substantially throughout the electrically resistant material.

A second example pattern 410 is shown in FIGS. 4(c)-(d). In this example, a single longitudinal array of sensors 412a-e may be used to measure the depth of delamination of an aluminum cantilever beam. As shown in FIG. 4(d), cantilever beam 416 has two cracks 414a-b. Sensor 412c may detect these cracks because the sensor 412c is likely to bend more than the other sensors due to the cracks. Thus, measurements of the electrical response of sensor 412c will indicate that sensor 412c is subjected to a greater strain than the other sensors (i.e., sensor 412c bends more than the other sensors). It should be appreciated that a similar situation may occur when a joint or bone is deteriorating. In an example, such a longitudinal arrangement may be helpful in identifying the beginning stages of a stress fracture. The longitudinal array may be useful for monitoring stress or strain behavior over a longitudinal portion of the body, such as the back, the spine, finger bones, leg bones (e.g., femur, tibia), or arm bones. As another example, the knee joint may be monitored using round-shaped flexible clothing to monitor joint bone-fluid conditions for all sides of the joint. Monitoring other joints using a longitudinal arrangement are possible as well.

Another example pattern is a circular array of thin-film sensors. A circular array may include thin-film sensors arranged in a circular, semicircular, or any round band. A circular pattern may be useful for monitoring stress or strain for an elbow, a knee, an ankle, or a wrist.

In example embodiment, the flexible thin-film sensor may be applied to any surface, including irregular and stretchable surfaces. That is, in addition to being used on smooth and flat surfaces, the flexible film may be used on any surface whose topology is not smooth and flat, as well as a surface of which is stretchable. The scale of the irregular surface is not a limiting factor for the thin-film sensor because the sensor is made from a thermosetting polymer as base matrix (e.g., epoxy). Hence, the sensor can be cast into any shape and size and applied onto any surface irrespective of its topology. Once the sensor is cast into a given shape, a conductivity in that state may be used as datum for the strain measurements. Generally, the sensor could be applied to any surface that is irregular in three dimensions. However, in an example, the sensor may not be applied to a surface having a sharp bend of more than approximately 270 degrees. Higher bending may cause additional straining due to the geometry effect and may possibly lead to an unexpected artifact in the signal.

In an example embodiment, the thin-film sensors may include approximately 33% CB by weight and 0.275-0.57% CNT by weight. However, different concentrations of materials are also possible. The concentration of these materials may vary, for example, depending on the desired application of the thin-film sensor. For instance, the preferred concentration of CB and CNTs may depend on the magnitude of strain being sensed and hence the magnitude of the load transfer. In an example, for applications that require a high degree of sensitivity, a greater concentration of CNT than 0.57% may be used to make the sensors highly sensitive (e.g., 3% CNT, 5% CNT, or 10% or more of CNT). High sensitivity may be useful to measure deformations in a patient who is experiencing the onset of pain due to minor bone deformations. Accordingly, sensors of a desired sensitivity may serve to provide an early indication of possible bone deformations.

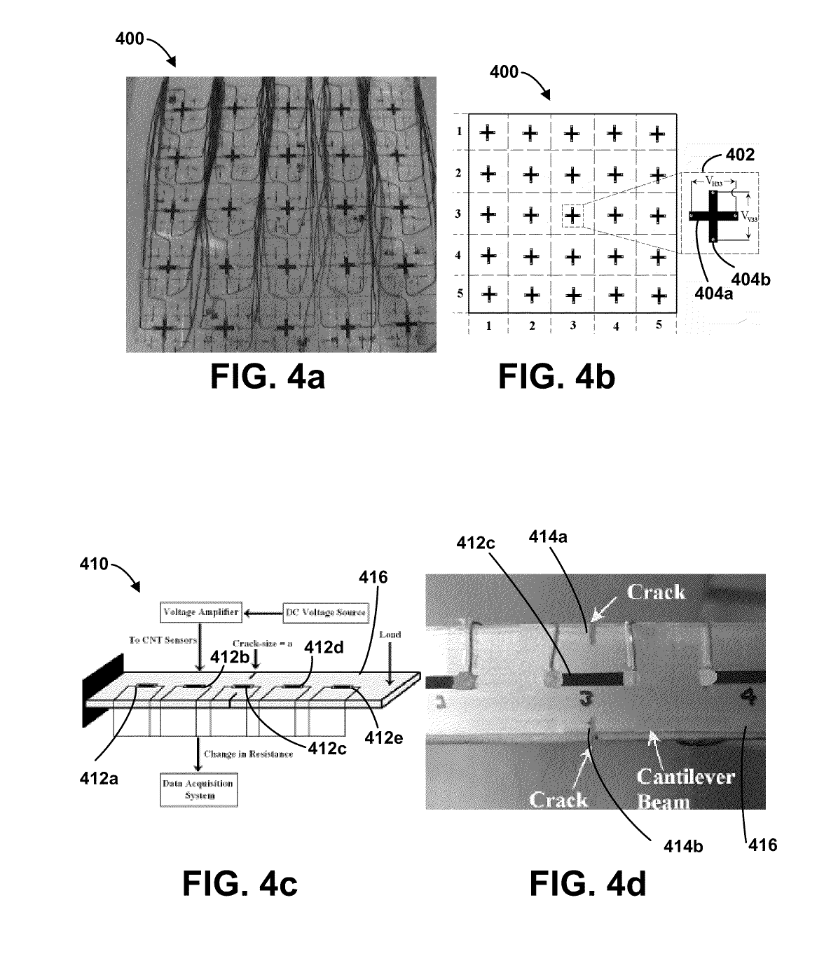

FIG. 5 depicts example thin film sensors including a fabricated composite of multiwalled carbon nanotubes, epoxy, and carbon black, in film and wire forms, on glass and polycarbonate substrates. In FIG. 5(a), a wire 502 made of a composite of multiwalled carbon nanotube, epoxy, and carbon black is mounted on a glass substrate 504. In FIG. 5(b), a multiwalled carbon nanotube, epoxy, and carbon black thin film 506 is mounted on a polycarbonate substrate 510. Polycarbonate substrate 510 may have a modulus of elasticity in the range of about 2-3 GPa. Conductive terminals such as a conducting terminal 508 on thin film 506 may be made from silver conducting glue or a similar substance.

The material of wire 502 may be filled into a thin mould on glass substrate 504, and the material may then be polymerized or cured. FIG. 5 shows wire 502 after the wire 502 was polymerized, for example. The material of film 506 can be filled into a shallow mould on polycarbonate substrate 510, and the material may be polymerized or cured, for example. FIG. 5 shows film 506 after the film 506 was polymerized, for example.

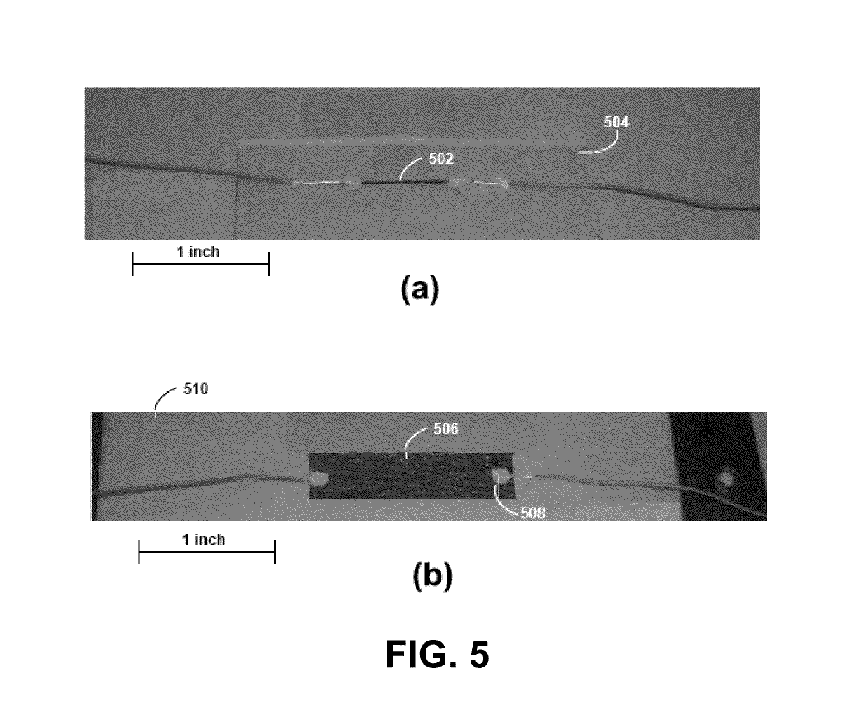

FIG. 6 is a block diagram that illustrates an example system 600 that may subject a thin film sensor to static loading, and includes a circuit that allows for measurement of current across the thin film sensor. A composite film 602, comprising an epoxy, carbon black, and carbon nanotube mixture, is connected via electrically conductive terminals 604 to a DC ammeter 606 and a DC voltage supply 608, to create a circuit. Composite film 602 may correspond to film 506 in FIG. 5, and conductive terminals 604 may correspond to conducting terminals 508. A clamping fixture 610 is mounted on a stable structure 612, and clamping fixture 610 anchors a substrate 614 to which composite film 602 is affixed. A tensile force 616 is applied to substrate 614, and by virtue of being affixed to substrate 614, composite film 602 experiences tensile force 616.

DC current voltage characterization may be measured to understand the electronic properties of the sensing film 602. The films prepared on the glass substrate may be connected to a DC power supply and an ammeter is connected in series, such as is shown in FIG. 6. The voltage may be varied continuously, and a corresponding current in the circuit may be measured. A temperature of the film 602 may be maintained to avoid temperature induced changes in the film 602, which may create an isothermal process. Strain may be applied on the film 602 by mechanically loading the film 602 as shown in FIG. 6.

Load may be applied in the length direction of the film 602 at a constant strain rate of about 0.5×10⁻⁴/s to create a quasi-static loading process. About 20 loading-unloading cycles may be carried out at the strain rate to allow stabilization of the film 602.

The film 602 may operate similar to a semiconductor under no applied mechanical load. With increasing voltage, current remains negligible until a certain bias voltage is reached, after which the current begins to increase steadily. Breakdown then occurs at a particular voltage, after which point the current increases rapidly.

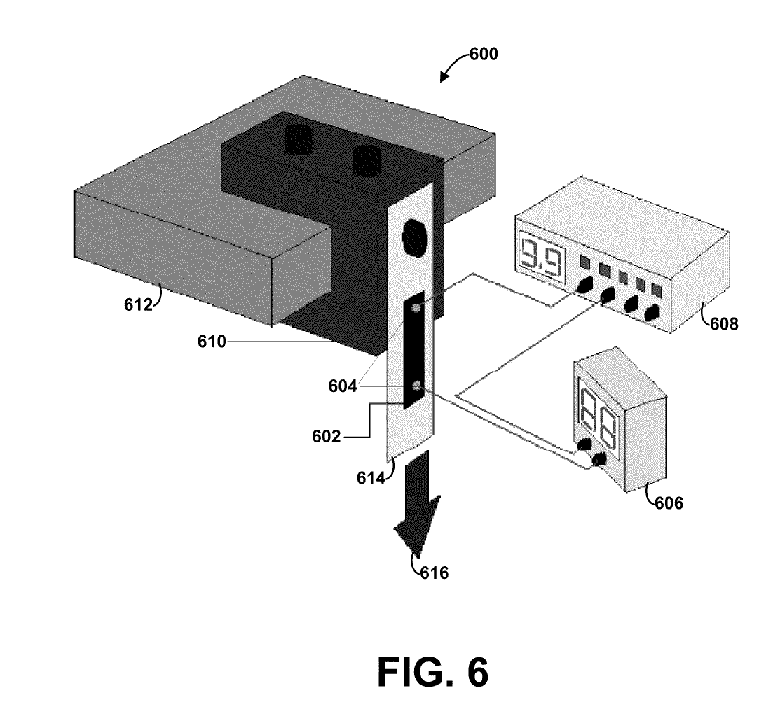

FIG. 7 is an example graph of experimentally-determined DC current voltage characteristics of carbon black and epoxy composites that have about 33% by volume carbon black under no mechanical load. For a wide range of voltages, from zero to about 500V, and across both polarities of voltage, the current response was approximately linear (and thus Ohmic) as shown in FIG. 7(a). However, at low magnitude voltages, from zero to a threshold voltage of about 9V, the carbon black and epoxy composite had little or no current response, as shown in FIG. 7(b). The carbon black and epoxy composite behaved similarly to a semiconductor diode, for example.

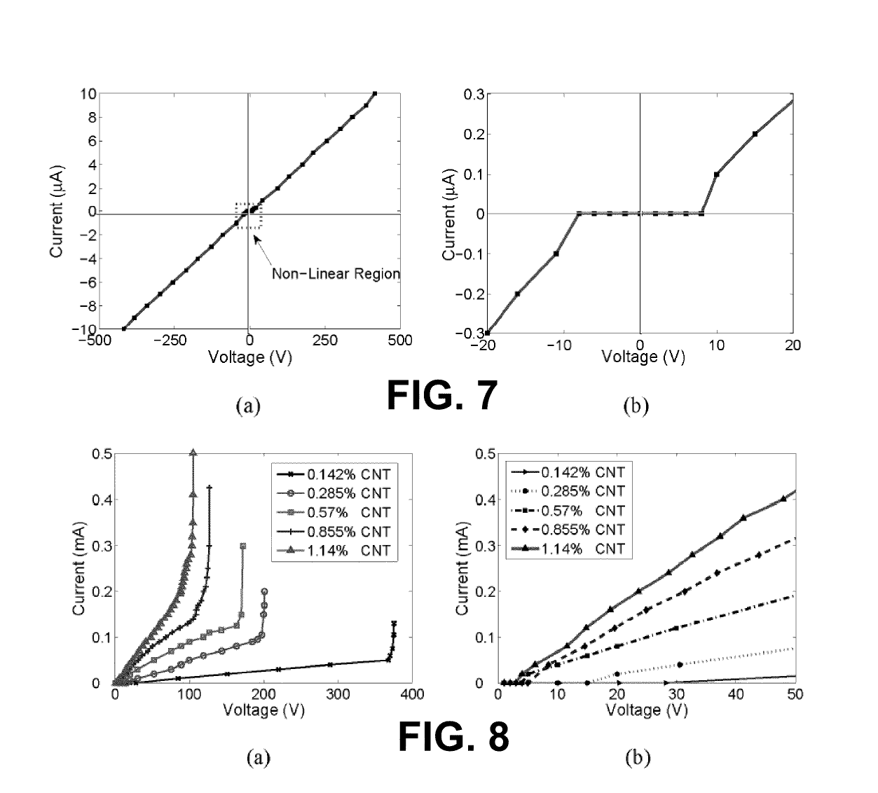

FIG. 8 is an example graph of experimentally-determined DC current voltage characteristics of epoxy, carbon black, and carbon nanotube composites that have about 33% by volume carbon black, and have various weight fractions of carbon nanotubes under no mechanical load. Five different weight fractions of carbon nanotubes were used with the following approximate weight fractions: 0.142%, 0.285%, 0.57%, 0.855%, and 1.14%. As shown in FIG. 8(a), a voltage at which the current began to increase rapidly decreased with an increase in carbon nanotube concentration. For example, at 0.142% CNT, the breakdown voltage was about 380V, and at 0.855% CNT, the breakdown voltage was only about 100V.

In addition, resistivity dropped with an increase in CNT concentration. This may be attributed to the creation of more conducting paths in the thin film due to the addition of CNTs. Similar to the CB/epoxy composites described in FIG. 7, the CNT/CB/epoxy films also showed a nonlinear behavior initially under no mechanical loading, as shown in FIG. 8(b). Current in all the films was negligible up to a certain voltage (e.g., the threshold voltage), after which point the current began to rise. The threshold voltage also decreased with the increase in the CNT concentration. For example, at 0.142% CNT, the threshold voltage was about 28V, and at 1.14% CNT, the threshold voltage was only about 3V. With the addition of 1% CNT by weight, the resistance of the composite film was reduced by an order of magnitude and the threshold voltage was reduced by over 90%, for example.

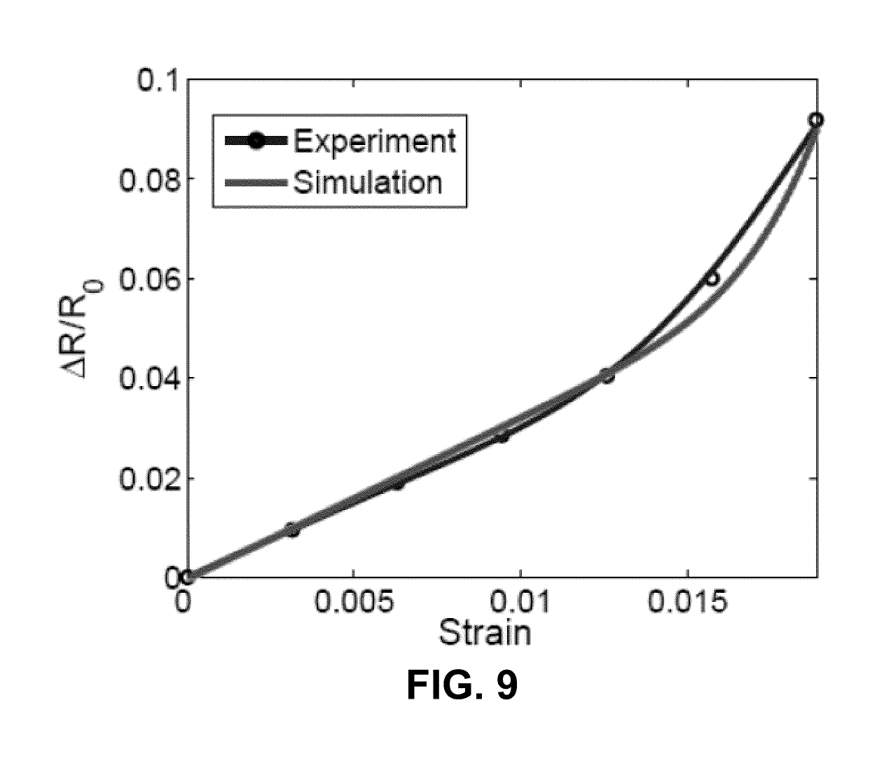

FIG. 9 is an example graph of a comparison between measured and simulated resistance change as a function of strain for a carbon black/epoxy composite that has about 33% by volume carbon black and no bias voltage. The composite showed a linear change in resistance up to a strain of about 1.2% and a nonlinear change for larger strains likely due to pre-yield softening of epoxy. The composite was sensitive to small strains and showed a resistance change of close to about 9% for a strain as small as about 2%, for example. The gauge factor was approximately 4.5, for example.

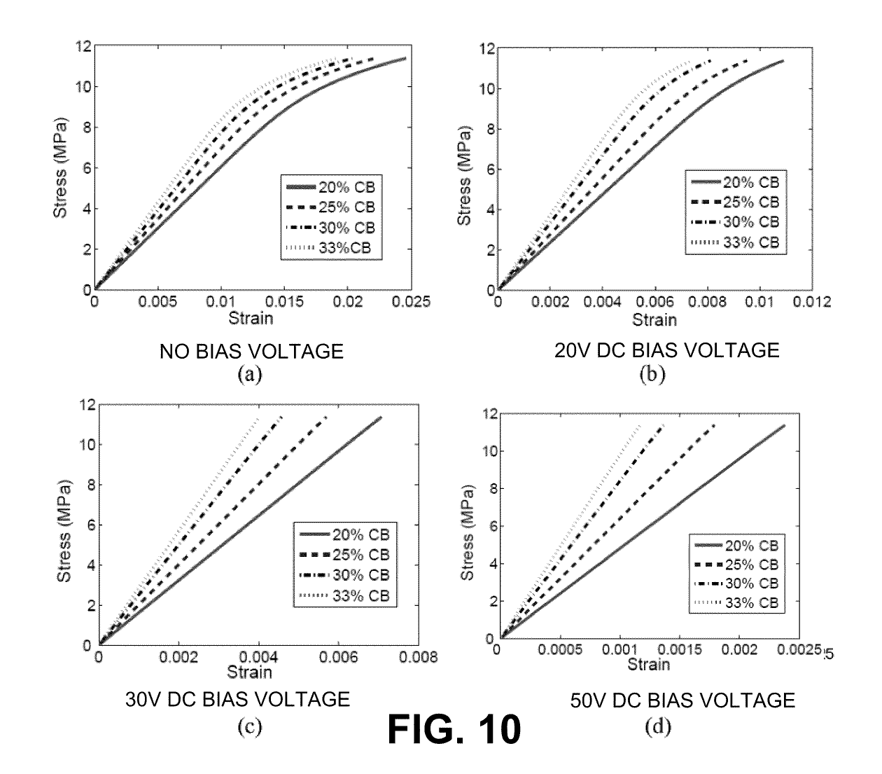

FIG. 10 is an example graph of simulated stress-strain curves for carbon black/epoxy films that have various volume fractions of carbon black at various bias voltages. The volume fractions of carbon black were about 20%, 25%, 30%, and 33%. The stress-strain behavior for all concentrations of carbon black was nonlinear when there is no applied bias voltage and at about 20V bias, as shown in FIG. 10(a)-(b). This may be attributed to the pre-yield softening of the composite films. The nonlinearity was reduced at higher bias voltages of about 40 and about 50V, as shown in FIGS. 10(c)-(d). This may be attributed to the polarization of the carbon atomic chains at higher voltages due to which the material started to stiffen. The stress-strain relationship strongly depended on the carbon black concentration and also the applied electric field. The stiffness of the composites increased with increases in both the filler concentration and the bias voltage. Under zero bias potential, the sample with about 33% by volume of carbon black has the highest stiffness, as seen in FIG. 10(a). Across all concentrations and bias voltages expressed in FIGS. 10(a) to 10(d), the sample with about 33% by volume carbon black at 50V bias voltage has the highest stiffness.

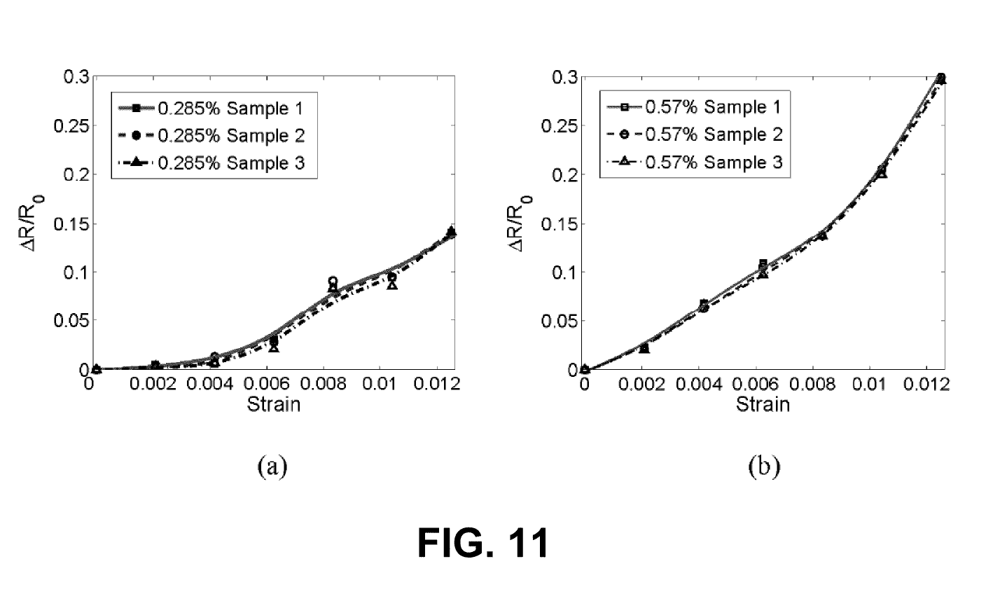

FIG. 11 is an example graph of experimentally-determined strain dependent resistance variations for sensors without an applied bias voltage and with different weight fractions of carbon nanotubes. For each of the weight fractions of about 0.285% CNT and about 0.57% CNT, three different samples of the same composition were tested, and the behavior was found to be similar for all the three samples. For strains ranging from 0-0.004, the gauge factor in the case of the film with 0.285% CNT in FIG. 11(a) was less than about 2. For strains over 0.01, the gauge factor in the case of the film with 0.285% CNT was between about 7 and 8. For strains ranging from 0-0.008, the gauge factor in the case of the film with 0.57% CNT in FIG. 11(b) was between about 5 and 7. For strains over 0.01, the gauge factor in the case of the film with 57% CNT was more than about 15. As can be seen in FIGS. 11(a) and 11(b), the gauge factor may increase with CNT concentration; the gauge factor roughly doubled from FIG. 11(a) to FIG. 11(B), as the CNT concentration roughly doubled. A gauge factor of a sensor may be expressed as an average of gauge factors of the sensor over a range of strain. An increase in gauge factor based on an increase in CNT concentration may be realized for concentrations of up to about 20% CNT, or even higher. In FIG. 11(b), the 0.57% CNT samples showed a resistance variation of about 30% at 2% strain which is a difference of almost 234% over a film with CB and epoxy but no carbon nanotubes, for example.

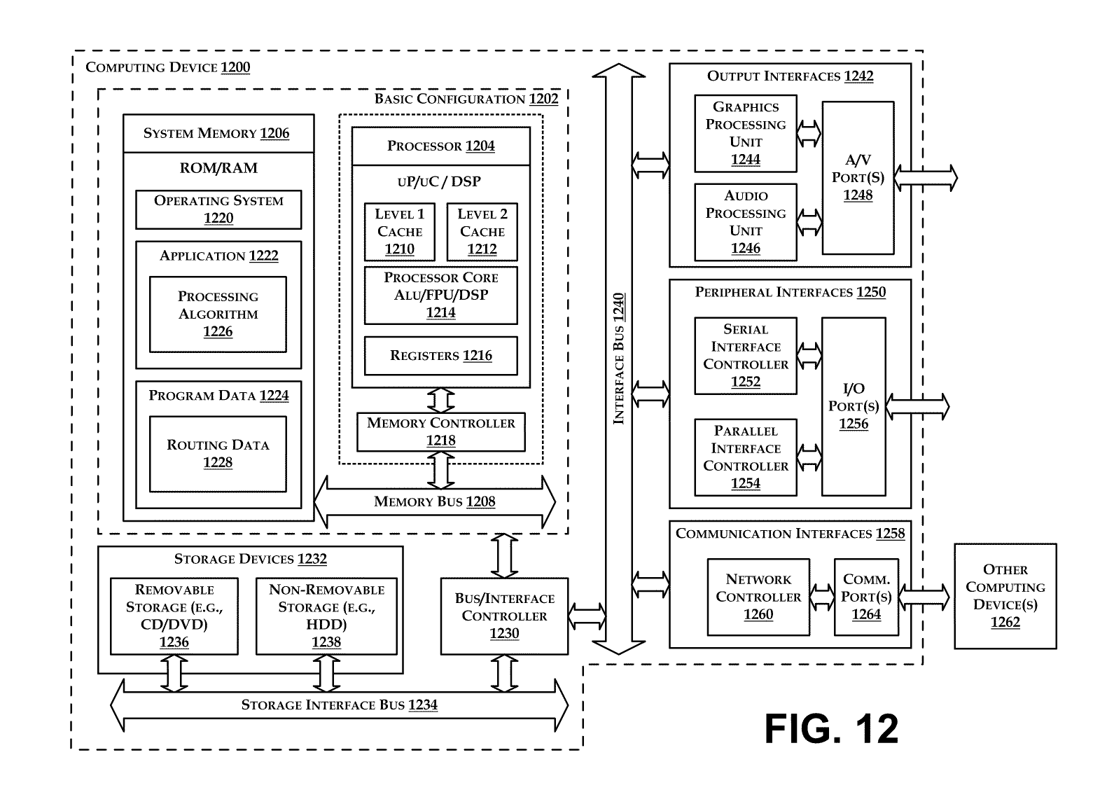

FIG. 12 is a block diagram illustrating an example computing device 1200, which may be a component of, a description of, or a system connected to example sensor apparatus 100 or data acquisition and analysis system 140. In a very basic configuration 1202, computing device 1200 typically includes one or more processors 1204 and system memory 1206. A memory bus 1208 can be used for communicating between the processor 1204 and the system memory 1206.

Depending on the desired configuration, processor 1204 can be of any type including but not limited to a microprocessor (μP), a microcontroller (μC), a digital signal processor (DSP), or any combination thereof. Processor 1204 can include one more levels of caching, such as a level one cache 1210 and a level two cache 1212, a processor core 1214, and registers 1216. The processor core 1214 can include an arithmetic logic unit (ALU), a floating point unit (FPU), a digital signal processing core (DSP Core), or any combination thereof. A memory controller 1218 can also be used with the processor 1204, or in some implementations the memory controller 1218 can be an internal part of the processor 1204.

Depending on the desired configuration, the system memory 1206 can be of any type including but not limited to volatile memory (such as RAM), non-volatile memory (such as ROM, flash memory, etc.) or any combination thereof. System memory 1206 typically includes an operating system 1220, one or more applications 1222, and program data 1224. Application 1222 includes algorithms 1226 that may be arranged to perform any function described herein depending on a configuration of the computing device 1200. Program Data 1224 includes data corresponding to the bits of a received preamble and routing data 1228. In some example embodiments, application 1222 can be arranged to operate with program data 1224 on an operating system 1220. This described basic configuration is illustrated in FIG. 12 by those components within dashed line 1202.

Computing device 1200 can have additional features or functionality, and additional interfaces to facilitate communications between the basic configuration 1202 and any required devices and interfaces. For example, a bus/interface controller 1230 can be used to facilitate communications between the basic configuration 1202 and one or more data storage devices 1232 via a storage interface bus 1234. The data storage devices 1232 can be removable storage devices 1236, non-removable storage devices 1238, or a combination thereof. Examples of removable storage and non-removable storage devices include magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media can include volatile and nonvolatile, removable and non-removable media implemented in any method or technology for storage of information, such as computer readable instructions, data structures, program modules, or other data.

System memory 1206, removable storage 1236 and non-removable storage 1238 are all examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 1200. Any such computer storage media can be part of device 1200.

Computing device 1200 can also include an interface bus 1240 for facilitating communication from various interface devices (e.g., output interfaces, peripheral interfaces, and communication interfaces) to the basic configuration 1202 via the bus/interface controller 1230. Example output interfaces 1242 include a graphics processing unit 1244 and an audio processing unit 1246, which can be configured to communicate to various external devices such as a display or speakers via one or more A/V ports 1248. Example peripheral interfaces 1250 include a serial interface controller 1252 or a parallel interface controller 1254, which can be configured to communicate with external devices such as input devices (e.g., keyboard, mouse, pen, voice input device, touch input device, etc.) or other peripheral devices (e.g., printer, scanner, etc.) via one or more I/O ports 1256. An example communication interface 1258 includes a network controller 1260, which can be arranged to facilitate communications with one or more other computing devices 1262 over a network communication via one or more communication ports 1264.

The communication connection is one example of a communication media. Communication media may typically be embodied by computer readable instructions, data structures, program modules, or other data in a modulated data signal, such as a carrier wave or other transport mechanism, and includes any information delivery media. A “modulated data signal” can be a signal that has one or more of its characteristics set or changed in such a manner as to encode information in the signal. By way of example, and not limitation, communication media can include wired media such as a wired network or direct-wired connection, and wireless media such as acoustic, radio frequency (RF), infrared (IR) and other wireless media. The term computer readable media as used herein can include both storage media and communication media.

Computing device 1200 can be implemented as a portion of a small-form factor portable (or mobile) electronic device such as a cell phone, a personal data assistant (PDA), a personal media player device, a wireless web-watch device, a personal headset device, an application specific device, or a hybrid device that include any of the above functions. Computing device 1200 can also be implemented as a personal computer including both laptop computer and non-laptop computer configurations.

As mentioned above, in example embodiments, the thin-film sensor has a long sensor lifetime. The lifetime of the sensor may depend on the usage and a number of cycles of usage. The lifetime of the sensor may be high, for example as high as a few million cycles. Such a lifetime is higher than the lifetime of existing sensors using only CNT or only CB as filler materials. These existing sensors suffer from low sensitivity and brittleness.

The CB/CNT/epoxy thin-film sensors may be used in a variety of applications. In addition to strain sensing for portions of a body of a patient (e.g., backs and joints), the sensor may be used conveniently in a number of other applications. For example, the sensor may be used in a bio-medical application. Example biomedical applications include strain sensing in implants and wearable devices and aiding device for physically challenged subjects. As another example, the sensor may be used in a defense and security application. Example defense and security applications include monitoring highly sensitive areas, intrusion detection using pressure fluctuation including battle-field and border surveillance, impact load sensing in armors and impactors.

As yet another example, the sensor may be used in a construction application. Example construction applications include structural health monitoring of critical structures and estimation of strain/stress pattern on critical structural elements. As yet another example, the sensor may be used in a sports application. Example sports applications include strain and stress in sports equipments and bio-mechanical reaction force monitoring for training of sports-persons. As yet another example, the sensor may be used in a consumer electronics application. Example consumer electronics applications include mechanical stress/strain and fatigue monitoring for critical microelectronic circuits and systems and monitoring and sensor feedback based control input in home appliances with moving parts.

As yet another example, the sensor may be used in a micro-electro-mechanical device application. Example micro-electro-mechanical device applications include micro and nano-scale strain engineering in devices and interconnects and polymer base layered architecture).

As yet another example, the sensor may be used in an automotive application. Example automotive applications include impact sensors, monitoring of axel loading, traction monitoring, human interface devices involving mechanical load application, and pressure sensitive coating.

As still yet another example, the sensor may be used in a haptics application. Example haptics applications include strain sensing for touch sensitive displays, coatings and wearable devices. Other applications are possible as well. From the above, it should be understood that the CB/CNT/epoxy thin-film sensor may be used in a wide variety of applications.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the teems of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method for identifying a strain history of a portion of a body of a patient, the method comprising:
measuring an electrical response of at least one thin-film sensor of a sensor apparatus that is applied to the portion of the body of the patient to obtain a reference signal, wherein the at least one thin-film sensor comprises an electrically resistant material, conductive nanoparticles dispersed substantially throughout the electrically resistant material, and conductive nano-structures dispersed substantially throughout the electrically resistant material, and wherein the at least one thin-film sensor has a gauge factor of greater than about 4;
monitoring the electrical response of the at least one thin-film sensor over a period of time to detect at least one change from the reference signal in the electrical response;
based on the at least one change from the reference signal in the electrical response, determining a strain history of the at least one thin-film sensor; and
identifying a strain history for the portion of the body of the patient based at least on the determined strain history of the at least one thin-film sensor.

2. The method of claim 1, wherein determining the strain history of the at least one thin-film sensor comprises (i) for each of a plurality of points in time over the period of time, determining a magnitude of strain of the at least one thin-film sensor at the point in time based on a difference between the electrical response at the point in time and the reference signal and (ii) compiling the determined magnitudes of the strain of the at least one thin-film sensor at the points in time to represent the strain history of the thin-film sensor.

3. The method of claim 1, further comprising:
comparing the identified strain history for the portion of the body of the patient to a pre-determined strain history;
based on the comparison, determining whether the portion of the body of the patient suffers from a medical issue related to the portion of the body.

4. The method of claim 3, wherein the portion of the body of the patient is a joint of the patient, and wherein the medical issue is a joint ailment.

5. The method of claim 4, wherein the joint ailment is an ailment selected from the group consisting of joint deterioration, arthritis, osteoporosis, plantar fasciitis, osteomalacia, and rickets.

6. The method of claim 1, wherein the at least one thin-film sensor of the sensor apparatus is disposed on a flexible membrane, and wherein the flexible membrane is attached to the portion of the body of the patient with an adhesive.

7. The method of claim 1, wherein measuring an electrical response of the at least one thin-film sensor to obtain a reference signal takes place under a no load condition.

8. The method of claim 1, wherein monitoring the electrical response of the at least one thin-film sensor over a period of time to detect at least one change from the reference signal in the electrical response comprises monitoring the electrical response of the at least one thin-film sensor in real-time.

9. The method of claim 1, wherein monitoring the electrical response of the at least one thin-film sensor over a period of time to detect at least one change from the reference signal in the electrical response comprises periodically measuring the electrical response of the at least one thin-film sensor.

10. The method of claim 1, further comprising tuning a sensitivity of the at least one thin-film sensor by adjusting a bias voltage applied to the at least one thin-film sensor.

11. The method of claim 1, wherein the conductive nanoparticles comprise amorphous carbon and the conductive nano-structures comprise carbon nanotubes.

12. A flexible sensor arrangement for identifying strain behavior for a portion of a body of a patient, the flexible sensor arrangement comprising:
a plurality of thin-film sensors, wherein each of the thin-film sensors comprise an electrically resistant material, conductive nanoparticles dispersed substantially throughout the electrically resistant material, and conductive nano-structures dispersed substantially throughout the electrically resistant material, wherein the thin-film sensor has a resistivity that varies with a magnitude of strain applied to the thin-film sensor,
wherein the plurality of thin-film sensors are arranged in a pattern for detecting strain behavior for the portion of the body of the patient.

13. The flexible sensor arrangement of claim 12, wherein the plurality of thin-film sensors are disposed on a flexible membrane, wherein the flexible membrane is attachable to the portion of the body of the patient.

14. The flexible sensor arrangement of claim 12, wherein the portion of the body of the patient is a joint of the patient.

15. The flexible sensor arrangement of claim 12, wherein each thin-film sensor is connected to a first electrical lead and a second electrical lead, and wherein the first electrical lead and the second electrical lead are connected a processing unit configured to measure an electrical response of the thin-film sensor.

16. The flexible sensor arrangement of claim 12, wherein the conductive nanoparticles comprise amorphous carbon and the conductive nano-structures comprise carbon nanotubes.

17. The flexible sensor arrangement of claim 12, wherein the pattern is a grid-like pattern of thin-film sensors.

18. The flexible sensor arrangement of claim 12, wherein the pattern is a longitudinal array of thin-film sensors.

19. The flexible sensor arrangement of claim 12, wherein the pattern comprises a circular array of thin-film sensors.

20. A sensor apparatus comprising:
a plurality of thin-film sensors, wherein each of the thin-film sensors comprise an electrically resistant material, conductive nanoparticles dispersed substantially throughout the electrically resistant material, and conductive nano-structures dispersed substantially throughout the electrically resistant material, wherein the thin-film sensor has a gauge factor of greater than about 4, wherein the plurality of thin-film sensors are arranged in a pattern for detecting stress behavior for the portion of the body of the patient;
a processing unit configured to measure electrical resistance of each of the plurality of thin-film sensors; and
a wireless communication interface in communication with the processing unit and arranged to transmit data from the processing unit.
